(19) United States
(12) Patent Application Publication
(10) Pub. No.: US 2026/0233230 A1
(43) Pub. Date: Aug. 13, 2026

(54) Container

(71) Applicant: Alphacath Limited, Ellesmere Port, Cheshire (GB)

(72) Inventors: Srinivasa Rao Vallabhaneni, Birkenhead (GB); Daniel Isler, Liverpool (GB); James Dickson, Liverpool (GB); Rafael Garcia-Figueras, Liverpool (GB)

(21) Appl. No.: 19/471,797

(22) PCT Filed: Apr. 12, 2024
(86) PCT No.: PCT/GB2024/050954

§ 371 Date: Oct. 2, 2025

(30) Foreign Application Priority Data
May 3, 2023 (GB) 2306509.7

(51) Int. Cl. B01L 3/00; A61B 10/00

(52) U.S. Cl. CPC B01L 3/508; A61B 10/0096; B01L 2200/141; B01L 2200/185; B01L 2300/043

(57) ABSTRACT

A container (10) which may be a delivery container and which has a container body (12) providing an internal volume (14) for receiving content. A content-insertion port 16 communicates with the internal volume (14) and is provided with a captor arrangement (17, 18). An access port (32) also communicates with the internal volume (14) and is provided with a cover (34) which is movable between open and closed configurations to open and close the access port. The captor arrangement (17, 18) is configured to permit insertion of content into the internal volume (14) via the content-insertion port but is configured to resist removal of content from the internal volume (14) by the same route. Content is instead removable from the internal volume (14) via the access port (32) when open.

The present invention relates to delivery containers.

When items are being transported it is often desirable to prevent or at least to resist unauthorised tampering. For example, where a filled medical-specimen receptacle is transported from a point of collection to a laboratory for analysis, the receptacle is often placed in some form of delivery container to provide it with protection on its journey. This delivery container may be as simple as a paper package lined with cushioning material such as bubble wrap, although a range of more substantial delivery containers is commercially available, often having a robust infection-moulded body to receive the specimen receptacle(s) and a lid to retain them. In the absence of some effective means for tamper prevention, there is the risk that a specimen could for example be substituted for another during its journey with malicious intent. There is also the possibility that samples might be inadvertently lost from the container, or that one sample might be accidentally substituted for another before the container is sealed for its journey.

So, a delivery container is needed that resists tampering with or substitution (be that deliberate or inadvertent) of its content.

The person despatching a package for delivery often cannot be assumed to have any specialist knowledge. For example, medical specimens may be collected by the patient at home and then posted or otherwise delivered to a laboratory for analysis. It is desirable that the delivery container should be easy to use, and that making the tamper resistant mechanism active should not require onerous user input, nor be reliant on a user remembering to activate it.

In accordance with the present invention there is a delivery container comprising:
a container body providing an internal volume for receiving content,
a content-insertion port communicating with the internal volume and provided with a captor arrangement,
an access port configured to be opened and closed,
   the captor arrangement being configured to permit insertion of content into the internal volume via the content-insertion port and being configured to resist removal of content from the internal volume via the content-insertion port, and content being removable from the internal volume via the access port when open.

Content such as a medical-specimen receptacle is thus easily insertable through the content-insertion port, and once inserted it is captive since the captor arrangement resists its withdrawal via the content-insertion port. Removal of the content from the container is effected by opening the access port. This may require a specific opening device—a key, a dedicated tool or a code. Hence unauthorised and/or unintentional removal of the content from the container is prevented, or at least resisted.

Specific embodiments of the present invention will now be described, by way of example only, with reference to the accompanying drawing, which depicts a set of delivery containers embodying the present invention.

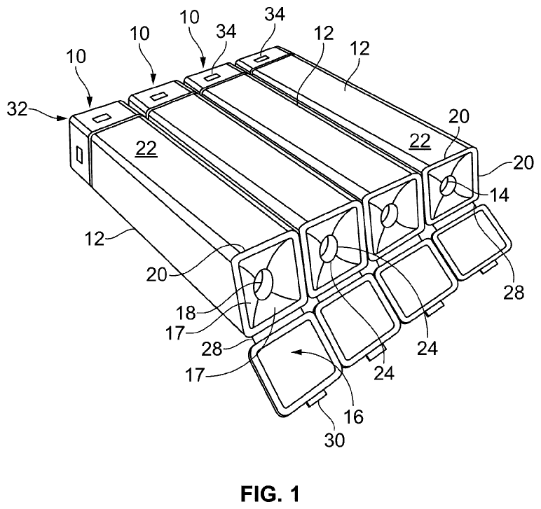

Delivery container 10 depicted in FIG. 1 is suited to use in delivery of medical specimen tubes, which are not depicted but which can take the form of a hollow cylindrical body provided with a screw top. Specimen tubes typically have an external label to carry information about the specimen.

The delivery container 10 comprises a hollow container body 12. In this example the container body 12 is of elongate cuboidal form, but it may take any number of different shapes without departing from the scope of the present invention. It may, for example, be cylindrical. Any suitable manufacturing technique and materials may be adopted but in the present example the container body 12 comprises moulded plastics material. Being hollow, the container body 12 defines an internal volume 14 (just visible in the drawing through an opening in the container) for receiving the intended content, which in the present case is the specimen tube.

The delivery container 10 is provided with a one-way content-insertion port 16 provided with a captor arrangement comprising a set of cantilevered fingers 17 arranged around a content-insertion opening 18. The captor arrangement facilitates insertion of the intended content to the internal volume 14 through the content-insertion port 16 but is configured to resist subsequent withdrawal of the content by the same route, thus rendering content in the internal volume captive. In the present embodiment the fingers 17 are resilient. Each finger 17 has a root 20 where it meets a wall 22 of the container body 12. Each finger 17 has a free end 24 and the free ends 24 together define the insertion opening 18, which in the present embodiment is round and has a diameter somewhat smaller than that of the specimen tube which is to be carried. The fingers 17 are each inclined inwardly, their free ends 24 being further into the internal volume 14 than their roots 20. The fingers 17 are resilient, being able to flex somewhat to change their angle of inclination, and subsequently to return to their original positions. The fingers 17 are roughly triangular in the present embodiment, so that arranged about a square end of the container body 12 they lie adjacent one another without overlap.

Insertion of the specimen tube into the container 10 simply involves pushing it through the insertion opening 18 of the content-insertion port 16. Because the specimen tube is oversize with respect to the insertion opening 18, the fingers 17 are thereby flexed to enlarge the opening 18. When the container 10 is fully inserted, the fingers 17 snap back to their original positions, and the specimen tube is then captive. The undersize insertion opening 18 means that anything one might insert to grip the specimen tube (be it human finger/thumb tips, say, or perhaps some form of gripping tool) would, when gripping the specimen tube, be too large to be easily withdrawn. But also due to the inclination of the fingers 17, their free ends tend, in response to anything being drawn outwardly through the insertion opening 18, to be driven toward one another, holding the item in question with a grip that tightens as the force exerted to try to withdraw it increases.

The container 10 may comprise internal dunnage (not shown) such as foam shaped to receive and embrace the specimen tube to protect it during its journey.

In the present embodiment a lid 26 is provided to close the insertion port 16. The lid is hinged to the container body 12, preferably through an integral (“living”) hinge 28 and is provided with a catch 30 to secure it in its closed position.

Removal of the specimen tube from the container 10 is provided for by means of an access port 32 through which access is gained to the internal volume 14. In the present embodiment the content-insertion port 16 is at one end of the container body 12 and the access port 32 is at its other end, but other layouts are possible without departing from the scope of the present invention. In FIG. 1 the access port is provided with a cover in the form of an end cap 34. To access the internal volume 14 the end cap 34 must be opened. For security the end cap 34 is provided with a locking mechanism. Opening it requires the use of an opener, whose form may vary from one embodiment to another. The opener may comprise a dedicated tool to mechanically release the end cap 34. It may comprise a key for receipt in a lock, opening which releases the end cap 34. It may comprise a code to be input to a mechanical lock, or to an electronically controlled lock. It may take other forms. In all such cases the opener may be made available at the container's destination—e.g. at the laboratory where a sample is to be analysed—but need not be made available at its point of origin, nor elsewhere. In this way a degree of protection is provided against unauthorised access to the content of the container 10 prior to its delivery.

As FIG. 1 shows, the containers 10 may be configured to engage one with another so that a plurality of them are able to be handled and transported as a single unit—the drawing includes four containers 10 which may be separated from one another.

The container 10 is well suited to repeated use. The environmental burden associated with single-use packaging can thus be avoided. It may be supplied to a user in a kit comprising, for example, a sample receptacle and the container 10. The container and the bottle may carry matching labels, as confirmation that they belong together.

The aforegoing embodiments are presented by way of illustration and not of limitation. Numerous variations may be made without departing from the scope of the present invention as set out in the appended claims. For example, the captor arrangement may in practice take an altogether different form, commensurate with its function of permitting insertion of content to the container's interior but resisting withdrawal of content by the same route.

1. A delivery container comprising:
a container body providing an internal volume for receiving content,
a content-insertion port communicating with the internal volume and provided with a captor arrangement,
an access port configured to be opened and closed,
the captor arrangement being configured to permit insertion of content into the internal volume via the content-insertion port and being configured to resist removal of content from the internal volume via the content-insertion port, and content being removable from the internal volume via the access port when open.

2. The delivery container as claimed in claim 1, wherein the access port is configured to be opened and closed by a cover which is lockable in a closed configuration, the cover being unlockable by use of an opener to enable the access port to be opened.

3. The delivery container as claimed in claim 2, wherein the opener comprises one of a key, a tool or a code.

4. The delivery container as claimed in claim 1, wherein the captor arrangement comprises a plurality of cantilevered fingers disposed around a content-insertion opening.

5. The delivery container as claimed in claim 4, wherein the fingers are inclined toward the internal volume and thereby configured to bend to admit content into the internal opening, and to grip content drawn through the content-insertion opening in a direction away from the internal volume.

6. The delivery container as claimed in claim 5, wherein the fingers each have a root through which the finger meets the container body, and a free end.

7. The delivery container as claimed in claim 6, wherein the free ends of the fingers together define a the content-insertion opening.

8. The delivery container as claimed in claim 7, wherein the content-insertion opening is substantially circular.

9. The delivery container as claimed in claim 1 provided with an internal dunnage to protect the content.

10. The delivery container as claimed claim 1, wherein the container body comprises two opposed ends, the content-insertion port being at one of the ends and the access port being at the other.

11. The delivery container as claimed in claim 1, further comprising a lid for closing the content-insertion port.

12. delivery system comprising content in a form of a receptacle and a delivery container as claimed in claim 1, wherein the delivery container configured for receiving the receptacle.

13. The delivery system as claimed in claim 12, wherein the receptacle is substantially cylindrical.

14. The delivery system as claimed in claim 12, wherein the receptacle is a medical specimen receptacle.
